(19) United States
(12) Patent Application Publication
(10) Pub. No.: US 2024/0257905 A1
(43) Pub. Date: Aug. 1, 2024

(54) DIFFERENTIATION OF COINFECTION FROM CONTAMINATION WITHIN GENETIC SAMPLES

(71) Applicant: Helix OpCo, LLC, San Mateo, CA (US)

(72) Inventors: Shishi Luo, San Francisco, CA (US); Alexandre Bolze, San Francisco, CA (US); Hang Dai, Mountain View, CA (US); Dana Wyman, San Diego, CA (US); Andrew Dei Rossi, San Carlos, CA (US)

(21) Appl. No.: 18/102,202

(22) Filed: Jan. 27, 2023

(51) Int. Cl. G16B 20/20; G16B 30/10

(52) U.S. Cl. CPC G16B 20/20; G16B 30/10

(57) ABSTRACT

Various embodiments disclosed relate to a method for detection of coinfection of two or more pathogen variants in an individual. The present disclosure includes methods of receiving a biological sample, selecting a first variant of the pathogen and a second variant of the pathogen, sequencing the biological sample, calculating a composite alternative allele fraction, and if the sample is a mixed sample, searching individual reads to detect a recombinant pathogen.

### TECHNICAL FIELD

This disclosure relates to bioinformatics, specifically to viral sequencing and analysis of viral variants.

### BACKGROUND

Viruses and other pathogens have a substantial impact on public health in all populations. Often, viruses can have several variants. Depending on the variant, the individual may be subject to different effects, and a preferred treatment plan may be altered. Moreover, determination of variants can help enhance epidemiological tracking of a virus or pathogen.

Determining specific variants of a virus responsible for infection in an individual can be complex. When a sample containing genetic material is collected from a patient, even when the genetic material is sequenced, a potential for misdiagnosis and other error is present. For example, where multiple variants are present in the sample, this may be the result of contamination of the sample. Contamination can be a problem resulting from more than one pathogen or pathogen variant in a sample.

However, in some cases, this may be the result of coinfection of the variants. While coinfection indicates that a patient hosted active copies of both variants at the same time, contamination indicates that samples with the variants may have been mixed outside of the patient.

Generally, laboratories collecting such samples discard samples showing signs of multiple variants, due to the possibility of contamination. While this reduces potential contamination, this approach also ignores potential coinfection of variants within an individual patient.

### SUMMARY OF THE DISCLOSURE

In an example, a method of distinguishing between coinfection or contamination for a biological sample having a first variant of a pathogen and a second variant of the pathogen, wherein the first variant corresponds to a first mutation and the second variant corresponds to a second mutation different than the first mutation, can include: acquiring sequencing data for the biological sample, the sequencing data including a plurality of reads; determining that the biological sample is a mixed sample of the first variant and the second variant based on an alternative allele fraction calculated according to the plurality of reads; searching individual reads of the plurality of reads for recombinant reads that include the first mutation and the second mutation; and determining whether the biological sample is indicative of a coinfection or a contamination, based on an amount of the recombinant reads that indicate the first variant and the second variant.

In an example, at least one non-transitory machine-readable medium can include instructions that, when executed by a processor of a machine, cause the processor to perform operations comprising: acquiring sequencing data for a biological sample, the sequencing data including a plurality of reads; identifying a first variant and a second variant of the biological sample, the first variant corresponding to a first mutation and the second variant corresponding to a second mutation different than the first mutation; determining that the biological sample is a mixed sample based on a composite alternative allele fraction; reviewing individual reads of the plurality of reads for recombinant reads including the first mutation and the second mutation; and determining whether the sample is indicative of a coinfection or a contamination.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a diagram of an example bioinformatics analysis system.

FIG. 1B is a flow chart illustrating a method of using a bioinformatics analysis system.

FIG. 2A is a flow chart illustrating a method of intaking and preparing a genetic sample in a wet laboratory for bioinformatic analysis in an example.

FIG. 2B is a flow chart illustrating a method of intaking a genetic sample in a wet laboratory for bioinformatic analysis in an example.

FIG. 2C is a flow chart illustrating a method of preparing a genetic sample in a wet laboratory for bioinformatic analysis in an example.

FIG. 3A is a flow chart illustrating a method of analyzing a genetic sample for contamination or coinfection with bioinformatic analysis in an example.

FIG. 3B is a flow chart illustrating a method of analyzing a genetic sample for contamination or coinfection with bioinformatic analysis using short reads in an example.

FIG. 3C is a flow chart illustrating a method of analyzing a genetic sample for contamination or coinfection with bioinformatic analysis using alternative allele fractions in an example.

FIG. 3D is a flow chart illustrating a method of analyzing a genetic sample for contamination or coinfection with bioinformatic analysis using short reads in an example.

FIG. 4 illustrates a schematic of a recombinant viral variant with a breakpoint in an example.

FIG. 5 depicts a computer system in an example.

FIG. 6 illustrates a graph of the spread of SARS-COV-2 lineages in the U.S. in an example.

FIG. 7A illustrates a schematic of a coinfection of two viral variants in an example.

FIG. 7B illustrates graphs of alternate allele fractions for mutations in an example.

FIG. 7C illustrates graphs of alternate allele fractions for mutations in an example.

FIG. 7D illustrates a comparison of variant fractions in an example.

FIG. 7E illustrates a graph of the dominant variant based on collection week in an example.

FIG. 7F illustrates a graph of viral load in coinfections in an example.

FIG. 8A illustrates evidence of recombinant read pairs in HMIX16 in an example.

FIG. 8B illustrates an IGV view of the alignments for HMIX16 in an example.

FIG. 9A illustrates a schematic of sample collection from a coinfection of two viral variants in an example.

FIG. 9B illustrates alternate allele fractions for mutations in an example.

FIG. 10 illustrates recombinant SARS-COV-2 reads in an example.

### DETAILED DESCRIPTION

### Process Overview

The present disclosure describes, among other things, methods of determining whether a biological sample is contaminated or indicates a coinfection.

Deoxyribonucleic Acid (DNA) viruses and Ribonucleic Acid (RNA) viruses can evolve to acquire a new combination of mutations through recombination, such as in adenoviruses. Recombination has also played an important role in the evolution of RNA viruses such as HIV-1 and SARS-COV-2 (“COVID”). Recently, as the COVID pandemic has progressed, the analysis of the SARS-COV-2 genomes showed many sequences of likely recombinant origins in patients.

However, tracking coinfections of the two variants has proved challenging. Such coinfections can be indicated by recombinant sequences for SARS-COV-2. Tracking these recombinant sequences is challenging due to the relatively low diversity of the genomes of the variants being recombined. Moreover, without the underlying sequencing data or orthogonal confirmation, it is difficult to determine whether recombinant sequences are real or are due to contamination, technical artifacts, or naturally occurring mutations shared by multiple variants.

The Omicron COVID variant, first detected by scientists in Botswana and South Africa in early November 2021, has rapidly spread across the globe. Notably, the Omicron variant is characterized by a large number of mutations in the spike protein thereon. In the U.S., the first Omicron case was reported in December 2017. At the time, the Delta COVID variant was dominant. Several Omicron sub-variants have since spread in the U.S. For example, Omicron BA. I was the dominant Omicron sub lineage initially in early 20121 while Omicron BA.2 began to rise in the United States in February 2022.

During the overlapping periods of Omicron and Delta infections in the U.S., a number of coinfections likely occurred and potentially resulted in the emergence of a new SARS-COV-2 variant resulting from the recombination of a Delta variant and an Omicron variant. This, in turn, would result in a new combination of mutations with unknown properties.

Thus, discussed herein are methods for analyzing cases of coinfection of DNA and RNA viruses (e.g., Delta and Omicron variants), and distinguishing such coinfection samples from contaminated samples. For example, the sample can be indicative of a pathogen having two variants. The first variant may have one or more markers distinguishable from one or more markers of the second variant. Here, a sample from a patient can be collected and analyzed for evidence of coinfection by the two pathogen variants. The biological sample can be collected (e.g., blood, saliva, etc.) and accessioned, amplified, then prepared for sequencing. The sample can then be short-read sequenced for bioinformatics analysis.

The sequencing data can include a plurality of reads, such as reads for shorter nucleic acid sequences that can be analyzed from 5′ end to 3′ end. These reads can be aggregated, and allele fractions calculated therefrom. If the composite Alternative Allele Fraction (AAF) is above a threshold, it is indicative that a single variant dominates the sample. However, if the AAF is below a threshold, it can be indicative that the sample includes genetic material from multiple variants of the pathogen.

To determine whether a mixed sample is a coinfection or contamination, after allele fraction analysis the individual reads can be analyzed and compared to the markers for the two variants of the pathogen. This can help determine if there is evidence of a recombinant pathogen, comprised of genetic material from both variants.

The present disclosure provides for rapid distinguishment between contaminated samples and samples representing a true coinfection. Discussed herein are methods for identifying and distinguishing contaminated samples from samples indicative of coinfection of multiple viral variants in an individual patient. Such samples can include genetic material from two variants of a pathogen.

In the case of contamination, such a sample may result from a liquid spill or other inadvertent mixing of material between samples. In contrast, coinfection samples are sourced from an individual suffering from infection of both variants. However, determining whether a sample containing two (or more) variants is contaminated or a true coinfection is challenging. For this reason, laboratories or bioinformatic analysts may disregard any samples with genetic material attributable to two or more variants, assuming the sample was contaminated.

The methods discussed herein distinguish between contaminated samples and coinfection samples by detecting evidence of a recombinant virus that includes genetic material from two variants at low but detectable frequencies. When detected, this result is consistent with template switching that occurs during replication in a cell infected with two or more variants. Template switching can be expected in many viruses, such as SARS-COV-2. For other pathogens, the recombination mechanism may differ.

Overall, evidence of a recombinant variant of a pathogen, made from genetic material of two different variants, is evidence that the two variants were actively replicating at the same time in a single cell. This is telling of a coinfection, as such a scenario does not occur where the sample has been contaminated in a laboratory. Samples in a laboratory that are contaminated are no longer biologically active.

The discussed methods provide a number of advantages, some of which are unexpected. The techniques can be configurable to avoid false positives caused by convergent evolution of different variants of a pathogen. This can be accomplished by only comparing variant-specific mutations and ignoring mutations that are common across multiple variants.

Additionally, the techniques can enable detection of which variant is dominant within a true coinfection. For example, once the first variant, second variant, and a recombinant variant are identified, a ratio or fraction of known variant-specific mutations can be found for each variant. Determining how the ratio changes across different portion of the genome for the pathogen can help identify the dominant variant.

### Definitions

As used herein, an “alternative allele fraction”, or an “alternate allele fraction” is the number of reads supporting an alternate allele (such as a mutation) divided by the total number of reads covering the position.

As used herein, an “average alternative allele fraction,” “median alternative allele fraction”, “mean alternative allele fraction”, or “weighted alternative allele fraction” is calculated respectively as the average, median, mean, or weighted value of alternate allele fractions at sites where at least a threshold, e.g., 15%, of the reads support a mutation. These terms may be generally referred to as “composite alternative allele fractions.”

As used herein, “accession” or “accessioning” refers to receiving and preparing a sample for later laboratory processes.

As used herein, “amplifying” refers to the production of multiple copies of a sequence of nucleic acid or other genetic material, such as RNA or DNA.

As used herein, “bioinformatics” refers to the science of collecting complex biological data such as genetic codes.

As used herein, “biological sample”, “sample” refers to a specimen from a patient, such as for bioinformatic research.

As used herein, “calling” an allele can include identifying one or more alleles, such as alternative alleles or mutations, at a particular locus of sequenced genetic material.

As used herein, “coinfection” or “co-infection” refers to a simultaneous infection of a host patient by multiple variants of a pathogen species.

As used herein, “contamination” refers to a sample that is impure, polluted, or unsuitable for biological analysis and research.

As used herein, “genetic material” refers to a fragment, molecule, or a group of nucleic acids, such as DNA or RNA, or other genetic material such as mitochondrial genetic material.

As used herein, “locus” or “loci” refers to the position of a gene or mutation on a chromosome or on a fragment of genetic material.

As used herein, “mixed sample” refers to a sample containing more than one variant of a pathogen.

As used herein, “mutation” refers to a changed structure of a gene that results in a variant form of the gene (e.g., with respect to a reference genome).

As used herein, “pathogen” refers to a bacterium, virus, or other microorganism that can cause disease.

As used herein, a “read” or “read pair” refers to data that defines a DNA or RNA sequence from one fragment or small section of genetic material.

As used herein, “recombinant” refers to genetic material formed by recombination (e.g., recombination of genetic material from two or more different variants).

As used herein, “sequencing” refers to a process of determining the nucleic acid sequence, the order of nucleotides in genetic material.

As used herein, “variant” or “genetic variant” refers to a subtype of a microorganism that is genetically distinct from other subtypes.

As used herein “walking” a sequence of genetic material can include reviewing and reading a sequenced portion of genetic material from the 5′ end towards the 3′ end to determine whether particular genetic markers, such as mutations, are present.

### Bioinformatic Analysis System and Methods

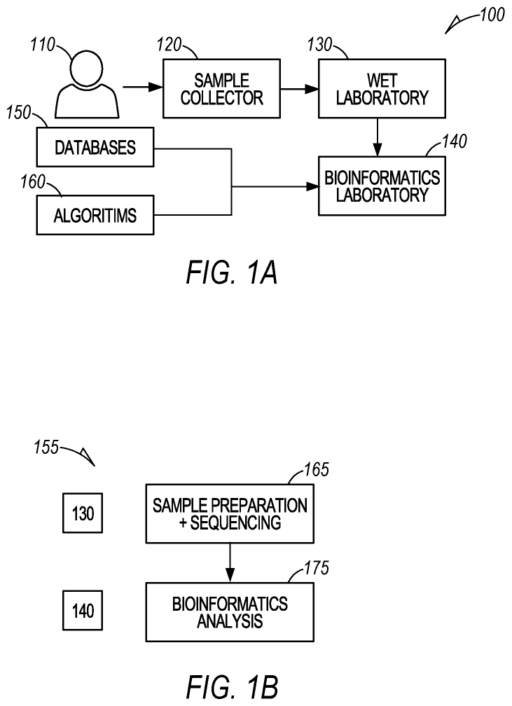

FIG. 1A depicts a diagram of an example bioinformatics analysis system 100, while FIG. 1B is a flow chart illustrating a method 155 of using such a bioinformatics analysis system 100.

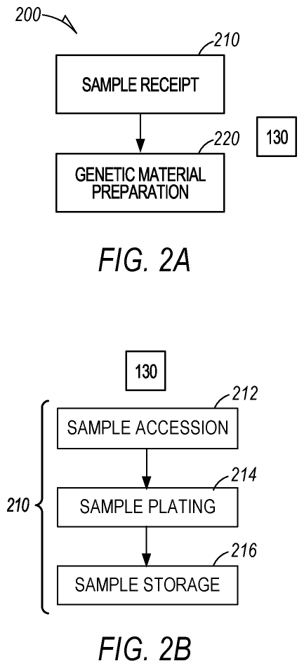

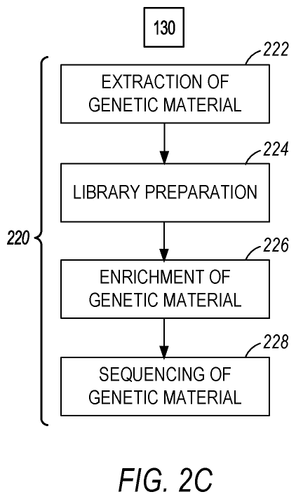

The system 100 can include both physical or “wet” laboratory components, and bioinformatics components. For example, the system 100 can interact with patients 110, from whom biological samples can be collected, in addition to sample collectors 120, which may be, for example, doctors, pharmacies, or other appropriate places where patient samples can be taken. The system 100 includes a wet laboratory 130 which is positioned to receive the biological samples and process those samples to produce sequenced genetic material for analysis, such as at step 165 of method 155. These methods of sample receipt, handling (e.g., accession), and sequencing, are discussed in detail below with reference to FIGS. 2A to 2C.

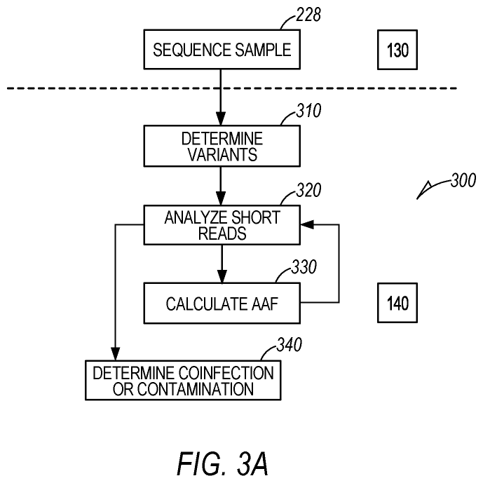

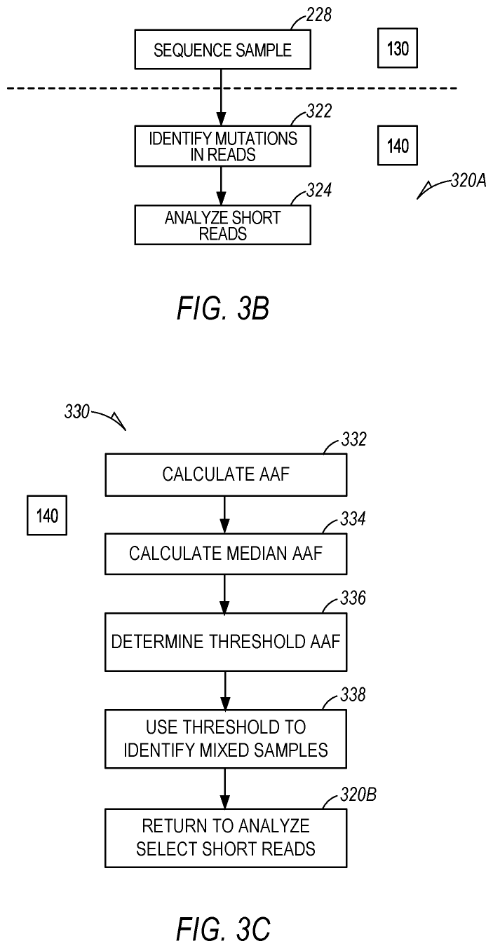

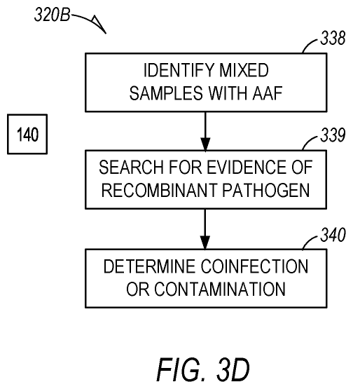

The system 100 can additionally include data driven components, such as databases 150 and algorithms 160 or other programs that support the bioinformatics laboratory 140 used to analyze genetic information. These data driven components can be used to do bioinformatic analysis (step 175 in method 155). Specific examples of such bioinformatic analysis are discussed in detail below with reference to FIGS. 3A to 3C.

### Sample Processing Methodology

Before bioinformatic analysis, biological samples are collected and sequenced through physical components of the system 100, such as through a wet laboratory 130. Methods of receiving and processing such samples are summarized in FIGS. 2A to 2C. FIG. 2A is a flow chart illustrating a method 200 of intaking and preparing a genetic sample in a wet laboratory for bioinformatic analysis. The method 200 can include two primary portions: receiving the samples (step 210) and preparing genetic material (step 220). FIG. 2B illustrates the first portion 210, including a method of intaking a genetic sample in a wet laboratory for bioinformatic analysis. FIG. 2C illustrates the second portion 220 of the method 200, a method of preparing a genetic sample in a wet laboratory for bioinformatic analysis.

The method 200 can begin with sample collection. For example, the samples can be collected by receiving a nasal swab, blood, saliva, or other material potentially containing genetic material indicative of a pathogen. The pathogen under study can be, for example, an RNA virus such as SARS-COV-2 or HIV, an adenovirus, or another type of pathogen with multiple variants having genetic material that could recombine for recombination and coinfection analysis.

Accessioning Samples. Once received at the laboratory, at step 212, the samples can be accessioned, that is, prepared for later laboratory processes. For example, accessioning can include receiving a batch of samples. A batch of samples can include, for example, hundreds of individual samples, or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a Quick Response (QR) code, a chip such as a Radio Frequency Identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or a pathogen, the external container can provide an additional barrier to protect technicians who may handle the samples. The external container can additionally include documentation correlating to the CSI, such as information on the patient that the sample was sourced from, information indicating circumstances of sampling, for example, a sampling date, a sampling method, a location that the sample was acquired, a name or title for a person who performed the sampling, other information, or combinations thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include Deoxyribonucleic Acid (DNA) or Ribonucleic Acid (RNA). In an example, the genetic material is one or more of many constituent components within the sample. For example, the genetic material may exist within the nuclei of white blood cells that are included within the sample. In another example, genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 50 minutes, or for another appropriate time period. In some cases, such as where the samples are the contents of a blood draw, the heating step may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, or is outside of an expected range of volume (e.g., between two and five milliliters), or other conditions, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database, and uniquely correlated to the CSI for the sample. The LSI can be associated with any error codes reported from the sample. Both the CSI and the LSI can both be applied to the sample container.

Sample Plating. Once accessioned, the samples can be plated at step 214. At this point, the sample have been successfully integrated into the laboratory environment and are ready for analytics. At this point, the samples can be prepared for transfer to a sample microplate. The sample microplate can be labeled with a unique identifier, which can distinguish the sample microplate from other sample microplates. For example, the sample microplate can be a solid body with about 50 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi-automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, and/or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at step 216. In some cases, accessioned samples, plated samples, or other samples, are stored for later use. In this case, they can be stored at room temperature, or can be cryogenically frozen and arranged on racks for later retrieval. Samples can be preserved for periods of days or years to allow later rapid re-testing.

Extraction of Genetic Material. When genetic analysis is desired, the genetic material of the samples can be extracted for sequencing at step 222. In some examples, a reagent can be applied to sample wells to lyse cells therein to expose genetic material.

Additionally, aspirating, and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing step can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at step 224. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome), may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well, and purification and/or elution of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at step 226 in FIG. 2C. This can enhance the ease of sequencing desired portions of the genome.

For example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, etc. That is, each probe can bind to a nucleic acid fragment at a specific location on the genome, and the range of probes can be selected to ensure that alleles, genes, the exome, or the entire genome of the subject being considered is acquired.

In these examples, utilizing probes in this manner can enhance efficiency of the sequencing process, by foregoing the need to sequence all of the roughly three billion base pairs found in the human genome. The enrichment process can further include controlling a concentration of the genetic material in each well, and purification and/or elution of the resulting material. Similar to the processes performed after extraction of genetic material, concentration of genetic material after enrichment can be confirmed for each well via testing.

Sequencing of Genetic Material. After enrichment, the genetic material can be sequenced at step 228. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier steps can be transferred to a flow cell, and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the library fragment can be washed away. Bridge amplification can then be performed to create copies of the remaining molecule in a localized cluster. For example, a cluster can comprise twenty to fifty copies of the same molecule, localized to a location the size smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence which can be measured. The emission wavelength and signal intensity for each cluster determines a base call for that cluster. A chemical group blocking a 3′ end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Depending on whether a complete human genome, or another set of genomic data, is being tested, different reagents can be chosen. That is, different reagents can be utilized for library preparation for a pathogen (e.g., bacteria, virus) or an organelle (e.g., mitochondria) than for a human genome. Pathogens exhibiting Ribonucleic Acid (RNA) genomes can have their genetic material translated to DNA before sequencing, enrichment, and/or library preparation are performed.

In some examples, genetic material can be used for detection of a pathogen rather than for sequencing. Detecting a pathogen can involve the use of a real-time PCR system that performs PCR. The real-time PCR system can further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen can thereby forego sequencing in some examples.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics such as quality indicator metrics. The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Determination of Coinfection or Contamination

After the samples have been received, processed, and sequenced in the wet laboratory environment 130 (see FIG. 1A and FIGS. 2A-2C), bioinformatic analysis can be done on the sequenced genetic material. An example method of bioinformatic analysis that involves determining whether a sample is indicative of a coinfection is shown and described with reference to FIGS. 3A to 3D.

Specifically, FIG. 3A is a flow chart illustrating a method 300 of analyzing a genetic sample for contamination or coinfection with bioinformatic analysis in an example. FIG. 3B illustrates analyzing short reads in a first instance of this method, FIG. 3C illustrates using alternative allele fractions in this method, while FIG. 3D illustrates analyzing short reads in a second instance in this method.

After being sequenced in step 228 as described above, the samples can be analyzed using bioinformatics in method 300 to determine whether the sample is indicative of a coinfection or contamination, such as by screening for recombinant genetic material of a pathogen in the samples.

In the method 300, variants of a pathogen can be identified (step 310). The sequenced genetic information (from step 228) and the identified variants (from step 310) can be used to analyze short reads (step 320) and to calculate alternative allele fractions (step 330). Both the alternative allele fractions (from step 330) and the analysis of short reads (from step 320) can together be used to determine whether a mixed sample is indicative of a coinfection or a contamination (at step 340). The steps 310 to 340 occur within the bioinformatics laboratory (140), that is, the data-driven space, not the wet laboratory (130).

In the method 300 for analyzing the sequence genetic material with bioinformatics to determine coinfection, two or more variants can first be determined (step 310). The genetic material that was sequenced (at step 228) from a sample includes sequences of a pathogen of interest. The pathogen can have multiple variants. Based, for example, on database information, two or more variants of the pathogen of interest can be identified at step 310. For example, in the case of SARS-COV-2, the first variant can be the Delta variant, and the second variant can be the Omicron variant.

Once the variants have been determined at step 310, short reads can be analyzed at step 320. Analysis of short reads (step 320) occurs twice in the method 300. First, shown in FIG. 3B, analysis of short reads (method 320A) can be done to identify mutations and call alleles corresponding to those mutations.

For the first variant, one or more mutations can be identified, and the corresponding allele called on the short reads. These can be mutation(s) that are unique to the first variant. Similarly, for the second variant, one or more mutations can be identified. These mutation(s) can be unique to the second variant of the pathogen. Such information can be acquired, for example, from one or more databases, such as database 150 in FIG. 1A, with which the bioinformatics laboratory 140 can be in communication. Such a database 150 can include historical or archive information related to pathogen variants.

Once the variants and corresponding mutations are identified, the short reads can be analyzed at method 320A shown in FIG. 3B. Analysis of the individual reads can be done in an automated or semi-automated manner.

Here, the sequenced genetic material from step 228 can include such short reads (e.g., the sequence length for each produced read is “short”, such as about 100 nucleotides to about 250 nucleotides). These short reads can be reviewed to find the relevant mutations (step 322) and call the corresponding alleles. The short reads can then be used in subsequent analysis (step 324). Using these short reads, the alternative allele fractions for each of the unique mutations can be determined.

FIG. 3C depicts a method 330 of determining whether a sample is a mixed sample using alternative allele fractions. The method 330 can include calculating an alternative allele fraction (step 332), calculating a composite alternative allele fraction (step 334), determining a threshold for the composite alternative allele fraction (step 336), identifying a mixed sample using the threshold (step 338), and returning to analyze selected short reads for mixed samples (step 340).

At step 334, the alternative allele fractions can be calculated. These methods can include determining the number of reads with the mutation (step 322 above) and using this information to calculate the alternative allele fraction (step 332).

In an example calculation of an alternative allele fraction, each of the mutations can be located within the genetic material at a particular locus between the 5′ end and the 3′ end on the read. For a specific mutation unique to one of the variants, an Alternative Allele Fraction (AAF) is the number of reads from the sample that support that mutation at that locus, divided by the total number of reads in the sample at that locus. An AAF can be calculated for each of the mutations of interest. In one embodiment, the composite alternate allele fraction is calculated as the median or mean value of alternate allele fractions at sites where at least 15% of the reads support a mutation.

At step 336, using the AAF, a composite AAF can be determined for the sample as a whole. A composite AAF can be calculated by determining the median or mean of the AAFs across loci of the pathogen which have an AAF of a threshold minimum amount. For example, where a specific mutation has been called at the locus, a composite AAF can be calculated at the locus using the reads where the AAF for that mutation at that locus is above about 0.15 or about 0.20.

At step 336, a desired threshold composite AAF value can be determined, outside of which the sample may be a mixed sample. At step 338, the determined threshold can be used to identify mixed samples.

Taking the calculated composite AAF, if a median or mean AAF at a particular locus is greater than a specific threshold, then the sample is likely dominated by a single variant and is neither contaminated nor a result of coinfection. For example, if the composite AAF at the locus is above about 0.80 or above about 0.85, it correlates to a large number of reads having that specific mutation at that specific locus, meaning one set of genetic material is present. In this case, the sample is consistent with a single variant infection. Thus, the sample is likely not representative of either coinfection or contamination.

Alternatively, if the composite AAF is less than the threshold, then this indicates that the sample may include genetic material from multiple variants of the pathogen; e.g., a “mixed sample.” A mixed sample may either be contaminated (such as due to a liquid spill) or may be evidence of a coinfection.

To determine whether the mixed sample has been contaminated or is from an individual with a coinfection, the sample reads are searched for evidence of a recombinant pathogen composed of genetic material from the two variants being considered. If a recombinant pathogen exists, this is evidence that a coinfection is present in the individual. A recombinant pathogen cannot be created by a liquid spill between samples, as such samples are biologically inert and hence are not capable of creating recombinant pathogens.

After identification of mixed samples (step 338) using alternative allele fractions, the individual short reads can be analyzed a second time (step 320B), as shown in FIG. 3D. Analysis of the individual reads can be done in an automated or semi-automated manner.

Further analysis of the short reads (method 320B) can be used to help determine whether the mixed sample is indicative of a coinfection or contamination. The method 320B can include identifying mixed samples with AAF (step 338), searching for evidence of a recombinant pathogen (step 328), and determining coinfection or contamination (step 340).

In an example, searching individual reads to detect a recombinant pathogen (step 328) can include identifying reads or read pairs straddling at least one of the first mutation and the second mutation. For example, reads or read-pairs straddling at least one variant-specific mutation for each of the variants being considered are identified. That is, two variant-specific mutations total, one for each variant, can be identified. For example, such a read-pair can begin before a variant-specific mutation for the first variant, and end after a variant-specific mutation for the second variant. If a read only straddles one variant-specific mutation, it is not sufficient to call it as recombinant.

In an example, a searching individual reads to detect a recombinant pathogen can include reviewing or “walking” across the sequence from the 5′ end to the 3′ end to determine whether the first mutation, the second mutation, or combinations thereof are present. This can also include identifying reads which start with the first mutation and send with the second mutation, in addition to identifying reads that include mutations from both the first variant and the second variant.

Each read can be walked across the sequence, such as from the 5′ end to the 3′ end of the read. Mutations that differ from a reference genome (that is, an originally detected variant of the pathogen) can be identified. If mutations 5′ to 3′ start out consistent with one viral variant, and then become consistent with another viral variant, this can be evidence of a recombinant pathogen. Many reads can support entirely one variant or the other.

However, if at least a threshold number of reads start with one variant, then have mutation indicated by another variant, then this is evidence that both variants were actively replicating within a live cell. Such a threshold number of reads can include, for example, about at least 0.05%, or at least about 0.01% of the reads, or at least about 2-3 reads, whichever is larger. Hence, the presence of two viral variants in the sample has not been caused by contamination, but rather by coinfection of the individual.

In an example, searching individual reads to detect a recombinant pathogen can include identifying one or more breakpoints. Further analysis can be performed to detect locations where reads for loci starting from a 3′ end of the genome have a ratio more than the threshold amount in favor of the first variant and reads for loci starting from a 5′ end of the genome have a ratio more than the threshold amount in favor of the second variant. The position(s) at which the reads start to call a different variant may then be identified as one or more breakpoints or breakpoint regions.

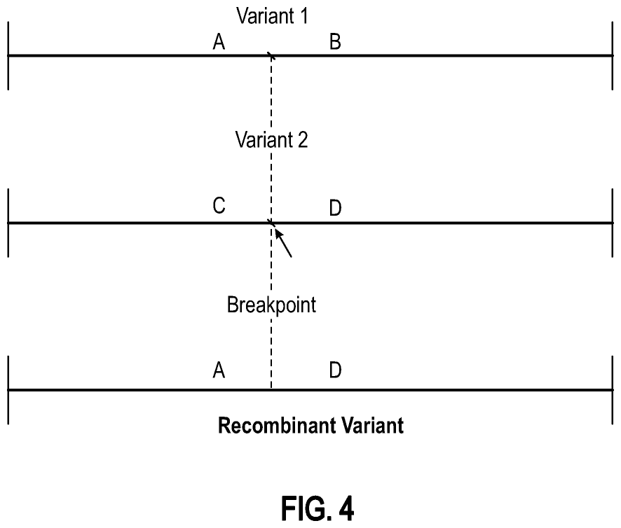

Visual example of such a break is illustrated in FIG. 4. Here, Variant 1 is shown having mutations A and B. Variant 2 is shown having mutations C and D. The Recombinant Variant is shown with mutations A and D, separated by the Breakpoint.

This technique can also be utilized to determine the dominant variant (i.e., the most common variant) within a coinfection. For example, a number of the chosen loci associated with the first variant, and a number of the chosen loci associated with the second variant, can be compared. In this case, a dominant variant can include a variant having a ratio higher than the threshold amount of reads for a chosen portion, for example at least about 60% or higher of the chosen loci.

This technique can also be used to identify co-infections, determine breakpoints, and determine the dominant variant resulting from three or more variants of a pathogen. In such an embodiment, so long as recombinant reads are detected that confirm each of the variants has encountered recombination, a corresponding sample may be determined to be the result of co-infection rather than contamination.

At step 340, based on the analysis of individual reads, a determination of coinfection or contamination can be made. After reviewing the reads, if the number of recombinant individual reads is sufficiently high, such as over a threshold, coinfection can be determined. Conversely, contamination can be determined.

If the individual is determined to be suffering from a coinfection, evidenced by the presence of a recombinant virus, then treatment for the individual can be modified to accommodate the presence of both variants. Examples of such treatments can include tailoring the various drugs and therapies to choose the most effective options for the patient. For example, monoclonal antibodies are less effective against Omicron BA.2 than they were against other variants. In this case, the doctor may change the treatment strategy in the case of an Omicron BA.2 and Delta coinfection.

Furthermore, sequence data may be utilized to determine a genome of a detected recombinant virus, benefitting the potential for tracking and treatment of the recombinant virus, should the recombinant virus become epidemiologically active.

### Computer Example

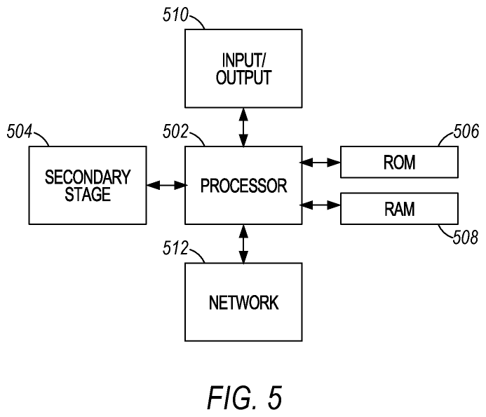

FIG. 5 illustrates a block diagram of an example computing system machine 500 upon which any one or more of the techniques (e.g., methodologies) discussed herein may be performed. Machine 500 (e.g., computer system) may include a hardware processor 502 (e.g., a central processing unit (CPU), a graphics processing unit (GPU), a hardware processor core, or any combination thereof), a main memory 504 and a static memory 506, connected via an interconnect 508 (e.g., link or bus), as some or all of these components may constitute hardware for systems 50 or 200 or hardware to operate the services and subsystems and related implementations discussed above.

Specific examples of main memory 504 include Random Access Memory (RAM), and semiconductor memory devices, which may include, in some embodiments, storage locations in semiconductors such as registers. Specific examples of static memory 506 include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; RAM; and CD-ROM and DVD-ROM disks.

The machine 500 may further include a display device 510, an input device 512 (e.g., a keyboard), and a user interface (UI) navigation device 514 (e.g., a mouse). In an example, the display device 510, input device 512 and UI navigation device 514 may be a touch screen display. The machine 500 may additionally include a mass storage device 516 (e.g., drive unit), a signal generation device 518 (e.g., a speaker), a network interface device 520, and one or more sensors 530, such as a global positioning system (GPS) sensor, compass, accelerometer, or some other sensor. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.). In some embodiments the hardware processor 502 and/or instructions 524 may comprise processing circuitry and/or transceiver circuitry.

The mass storage device 516 may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the mass storage device 516 constitutes, in at least some embodiments, machine readable media.

The term “machine readable medium” includes, in some embodiments, any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Specific examples of machine-readable media include, one or more of non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; RAM; and CD-ROM and DVD-ROM disks. While the machine readable medium 522 is illustrated as a single medium, the term “machine readable medium” includes, in at least some embodiments, a single medium or multiple media (e.g., a centralized or distributed database, and/or associated caches and servers) configured to store the one or more instructions 524. In some examples, machine readable media includes non-transitory machine-readable media. In some examples, machine readable media includes machine readable media that is not a transitory propagating signal.

The instructions 524 are further transmitted or received, in at least some embodiments, over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, a Long Term Evolution (LTE) 4G or 5G family of standards, a Universal Mobile Telecommunications System (UMTS) family of standards, peer-to-peer (P2P) networks, satellite communication networks, among others.

An apparatus of the machine 500 includes, in at least some embodiments, one or more of a hardware processor 502 (e.g., a central processing unit (CPU), a graphics processing unit (GPU), a hardware processor core, or any combination thereof), a main memory 504 and a static memory 506, sensors 530, network interface device 520, antennas 532, a display device 510, an input device 512, a UI navigation device 514, a mass storage device 516, instructions 524, a signal generation device 518, and an output controller 528. The apparatus is configured, in at least some embodiments, to perform one or more of the methods and/or operations disclosed herein. The apparatus is, in some examples, a component of the machine 500 to perform one or more of the methods and/or operations disclosed herein, and/or to perform a portion of one or more of the methods and/or operations disclosed herein.

In an example embodiment, the network interface device 520 includes one or more physical jacks (e.g., Ethernet, coaxial, or phone jacks) or one or more antennas to connect to the communications network 526. In an example embodiment, the network interface device 520 includes one or more antennas 532 to wirelessly communicate using at least one of single-input multiple-output (SIMO), multiple-input multiple-output (MIMO), or multiple-input single-output (MISO) techniques. In some examples, the network interface device 520 wirelessly communicates using Multiple User MIMO techniques. The term “transmission medium” shall be taken to include any intangible medium that is capable of storing, encoding or carrying instructions for execution by the machine 500, and includes digital or analog communications signals or other intangible medium to facilitate communication of such software.

At least some example embodiments, as described herein, include, or operate on, logic or a number of components, modules, or mechanisms. Such components are tangible entities (e.g., hardware) capable of performing specified operations and are configured or arranged in a certain manner. In an example, circuits are arranged (e.g., internally or with respect to external entities such as other circuits) in a specified manner as a module. In an example, the whole or part of one or more computer systems (e.g., a standalone, client or server computer system) or one or more hardware processors are configured by firmware or software (e.g., instructions, an application portion, or an application) as a component that operates to perform specified operations. In an example, the software resides on a machine-readable medium. In an example, the software, when executed by the underlying hardware of the component, causes the hardware to perform the specified operations.

Accordingly, such components are understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which components are temporarily configured, each of the modules need not be instantiated at any one moment in time. For example, where the components comprise a general-purpose hardware processor configured using software, in some embodiments, the general-purpose hardware processor is configured as respective different components at different times. Software accordingly configures a hardware processor, for example, to constitute a particular component at one instance of time and to constitute a different component at a different instance of time.

Some embodiments are implemented fully or partially in software and/or firmware. This software and/or firmware takes the form of instructions contained in or on a non-transitory computer-readable storage medium, in at least some embodiments. Those instructions are then read and executed by one or more hardware processors to enable performance of the operations described herein, in at least some embodiments. The instructions are in any suitable form, such as but not limited to source code, compiled code, interpreted code, executable code, static code, dynamic code, and the like. Such a computer-readable medium includes any tangible non-transitory medium for storing information in a form readable by one or more computers, such as but not limited to read only memory (ROM); random access memory (RAM); magnetic disk storage media; optical storage media; flash memory, etc.

Various embodiments may be implemented fully or partially in software and/or firmware. This software and/or firmware may take the form of instructions contained in or on a non-transitory computer-readable storage medium. Those instructions are then read and executed by one or more processors to enable performance of the operations described herein. The instructions are in any suitable form, such as but not limited to source code, compiled code, interpreted code, executable code, static code, dynamic code, and the like. Such a computer-readable medium includes, in at least some embodiments, any tangible non-transitory medium for storing information in a form readable by one or more computers, such as but not limited to read only memory (ROM); random access memory (RAM); magnetic disk storage media; optical storage media; flash memory, etc.

### EXAMPLES

Various examples of the present disclosure can be better understood by reference to the following Examples which are offered by way of illustration. The present disclosure is not limited to the Examples given herein.

### Example 1. SARS-COV-2 Delta and Omicron Variant Sample Collection and Sequencing

Between November 2021 and February 2022, SARS-COV-2 Delta and Omicron variants co-circulated in the United States, allowing for coinfections of patients, and potentially recombinant events within patients infected by both variants. Discussed in the Examples here, 29,719 positive COVID samples were received and analyzed during this time frame to detect which variant was present. Within these samples, the presence (or fraction of reads) of mutations specific to either the Delta variant or the Omicron variant supported the occurrence of the respective variant.

The 29,719 samples were collected across the United States between Nov. 22, 2021, and Feb. 13, 2022.

The samples were anterior nasal swabs of individual patients. The viral samples were collected through the Helix® Opco diagnostic testing laboratory and run through the standard test processing workflow. The test was based on the Thermo Fished TaqPath COVID-19 Flu A, Flu B Combo Kit, which targets three respiratory pathogens (SARS-COV-2, Influenza A, and Influenza B). Nasal swab samples were transported in saline and sample tubes were heat inactivated upon receipt at the lab. Test results from positive cases, in addition to metadata including sample collection date, state, and qRT-PCR Cq values were used to build the database in these Examples.

A single viral sequence assay was performed for each person infected. The majority of samples were collected from a national retail pharmacy. A portion of the samples were collected from San Diego County, as part of community testing organized there. As summarized in Table 1 above, the individuals tested represented a diverse range of race, age and gender groupings.

The samples were sequenced and assigned a lineage. For sequencing, RNA was extracted from 400 μL of patient anterior nares sample using the MagMAX Viral/Pathogen kit (ThermoScientific). Sequencing libraries were generated with total RNA library preparation (5 μL RNA input volume) using the Rapid RNA Library Kit protocol (Swift Biosciences/Integrated DNA Technologies). SARS-COV-2 genome capture was accomplished using hybridization kit xGen COVID-19 Capture Panel (Integrated DNA Technologies). Samples were sequenced using the NovaSeq 6000 Sequencing system S1 flow cell, with S1 Reagent Kit v1.5 for 300 cycles. The sequence data was uploaded for bioinformatic processing.

The sample data was processed for further sequencing and lineage assignment. The flow cell output was demultiplexed with bcl2fastq (Illumina) into per-sample FASTQ sequences that were then run through a fast generator pipeline (Helix) to produce a sequence FASTA file. Subsequently, sequence reads were aligned to a reference comprising the SARS-CoV-2 genome (NCBI accession NC_045512.2) and the human transcriptome (GENCODE v37) using BWA-MEM. Reads were then marked for duplicates before proceeding to variant calling using the Haplotyper algorithm (Sentieon, Inc).

Finally, the per-base coverage from the alignment file (BAM) and per-variant allele depths from the variant call format (VCF) file were used to build a consensus sequence. The following criteria were used: coverage from at least 5 unique reads was required with at least 80% of the reads supporting the allele. Otherwise, that base was considered uncertain, and an “N” was reported.

Quality control (QC) of the viral sequences occurred at two levels: sample and plate. A sample-level QC status of ‘pass’ indicated a sample was unlikely to have been contaminated and had a sufficiently complete consensus sequence to be assigned a lineage. For a qc_status of ‘pass’, a sample required a composite alternate allele fraction of at least 0.8 for its variants (any variant VCF record in the Haplotyper VCF file) and a consensus sequence containing at most 30% N bases. At the plate level, the QC criteria were designed to flag potential reagent issues or sample swaps that would require an entire plate to be re-processed.

Viral sequences were assigned a Pango lineage using pangoLEARN. For this analysis, pangoLEARN version 2022-02-02 with Pangolin software version 3.1.11 was used. 29,719 sequences from samples collected between Nov. 22, 2021, and Feb. 13, 2022, for genomic surveillance purposes were sequenced attributed a lineage.

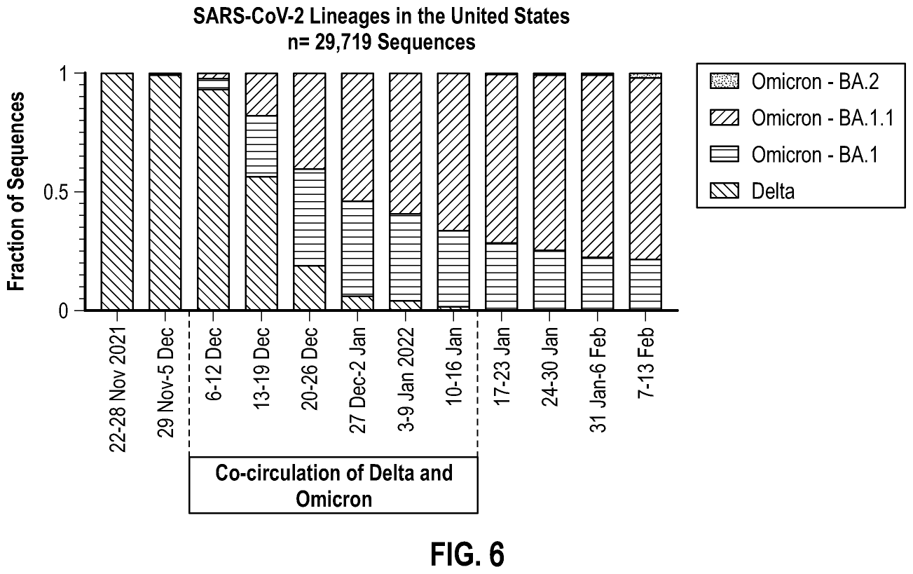

As shown in FIG. 6, the Omicron variant quickly grew to explain >99% of cases as of the week of January 17th. FIG. 6 depicts co-circulation of Delta and Omicron variants in the United States. Fraction of lineages sequenced per week in the United States. Delta includes B.1.617.2 and all lineages starting with AY. The week of collection of the sample is on the X-axis.

Thus, Delta and Omicron variants co-circulated (representing greater than 1% of infections) from Dec. 6, 2021, to Jan. 16, 2022, represented by 14,214 sequences in shown in FIG. 6 amongst the dataset analyzed herein.

During that time, the overall number of cases in the United States remained high, above 150,000 new cases per day and above a 7-day case rate of 250 per 100,000 individuals. Thus, possibility of a coinfection by two distinct variants high during this period.

The samples, once collected, sequenced, and assigned a lineage, were further analyzed for determination of coinfections of Delta and Omicron variants as discussed below with reference to the Examples.

### Example 2. Investigation of Coinfection of Delta and Omicron Variants with Genotyping and Allele Fractioning

After collection and sequencing, the samples were further analyzed for coinfection with Delta and Omicron variants. When a patient is infected by two distinct variants, such as the Delta and Omicron variants of the SARS-COV-2 virus, multiple copies of the full genome of each variant are present in the sample. A fraction (x %) of the total extracted SARS-CoV-2 RNA came from Delta, and the remaining fraction (100-x %) of the RNA will come from Omicron.

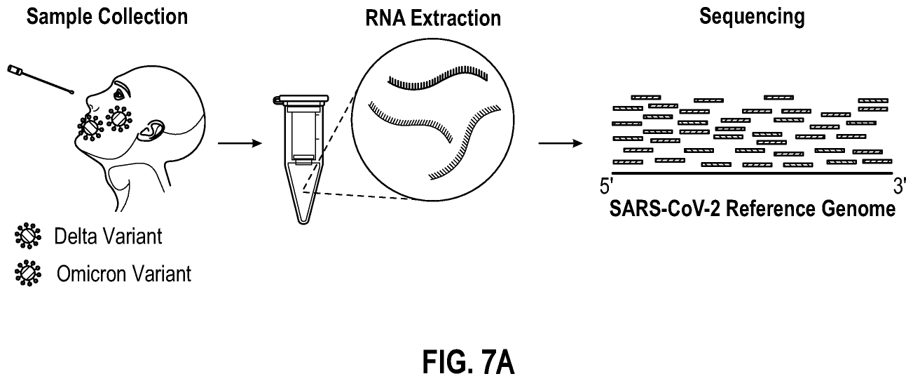

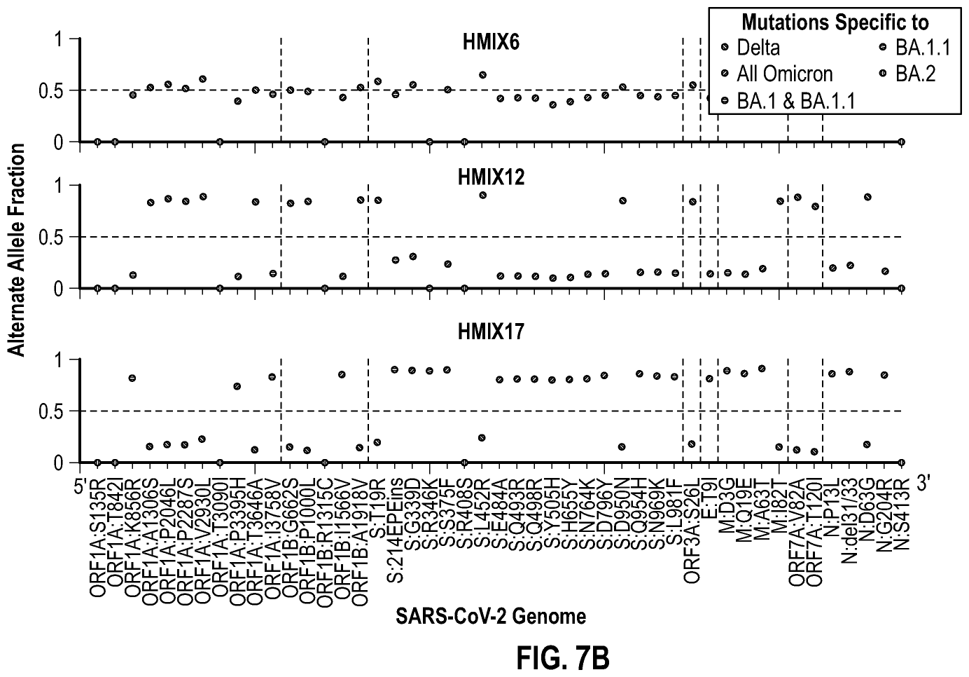

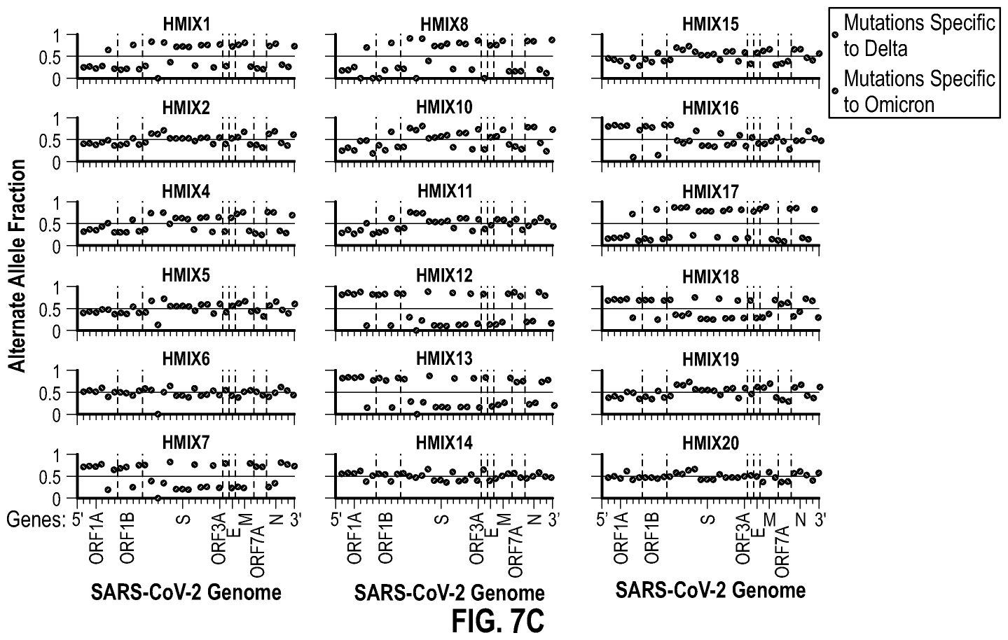

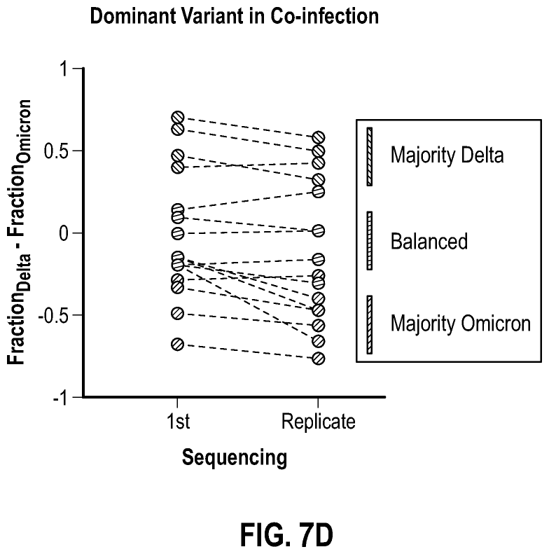

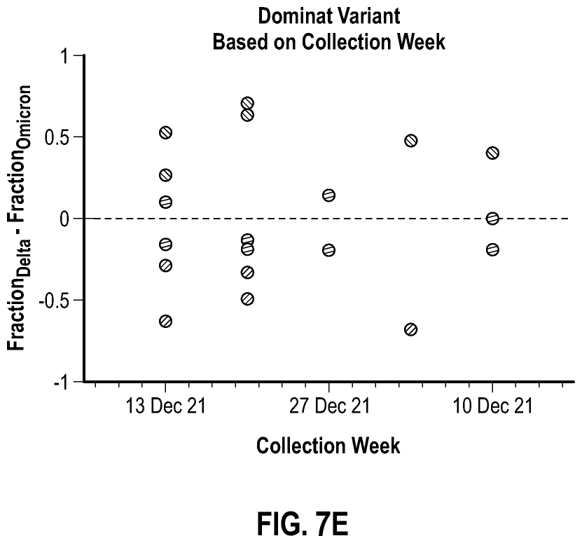

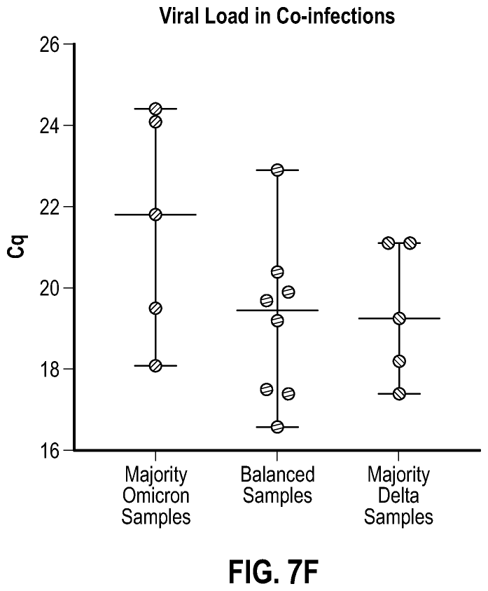

Sequencing at a high enough coverage led to calling mutations that defined both Delta and Omicron, but each mutation was only be supported by a fraction of the reads overlapping the given position. For example, the mutations specific to Delta were called with ˜x % of the reads overlapping the position, whereas the mutations specific to Omicron were called with (100-x)%, as shown in FIG. 7A, which depicts a schematic of a coinfection and the impact on the sequencing output.

Thus, in this Example, to identify this coinfection signature, a list of mutations specific to the Delta variant was selected, and a list of mutations specific to the Omicron variant was selected. The four specific markers used when looking at Delta and Omicron variants, shown in Table 2:

TABLE 2
Viral variants and markers.

| Variant | Sub-Variant | Marker |
|---|---|---|
| Delta | — | C21618G |
| Omicron | BA.1 | G8393A |
| Omicron | BA.1 | T13195C |
| Omicron | BA.1 | C23202A |

The mutations selected had a call (not an “N”) in >95% of the samples between November 2021 and February 2022.

The relative allele fraction of each variant in coinfections was investigated. The number of RNA copies and coverage varies across the SARS-COV-2 genome. Moreover, the density of Delta and Omicron mutations varies. For example, there are more Omicron-specific mutations in the spike protein.

Thus, in this Example, four Delta-specific mutations and four Omicron-specific mutations, spread across the SARS-COV-2 genome, were used to calculate the mean allele fractions for each variant in each sample. These are summarized below in Table 3:

TABLE 3
Viral mutations.

| Variant | Mutations |
|---|---|
| Delta | ORF1A:P2046L * |
| | ORF1B:P1000L |
| | S:T19R |
| | M:I82T |
| | *Not a defining mutation of Delta, but present in all |
| | coinfection samples of this study |
| Omicron | ORF1A:P3395H |
| | ORF1B:I1566V |
| | S:N969K |
| | M:A63T |

Allele fractions of the samples were calculated as discussed above. Based on the calculated allele fractions, Delta was considered the dominant variant if the Delta fraction was above 60% and the Omicron fraction was below 40%. Omicron was considered the dominant variant if the Omicron fraction was above 60% and the Delta fraction was below 40%. Other samples were considered balanced. The results of the initial sequencing were used to decide which variant was dominant for each sample shown in Table 4:

TABLE 4
Samples with coinfections and Delta-Omicron infections

| | | | | Unique | | | | | | |
|---|---|---|---|---|---|---|---|---|---|---|
| | | | | SARS- | | | | | | |
| | Collect | | COVID | CoV-2 | Median | | | Replication | Dominant | Break |
| Name | week | State | Cq | reads | AAF | Lineage | Clade | method | variant | point |
| HMIX1 | 2021 Dec. 20 | CA | 18.1 | 480,979 | 0.71 | None | 21K | Re- | Omicron | N/A |
| | | | | | | | (Omicron) | extraction | | |
| | | | | | | | | and re- | | |
| | | | | | | | | sequencing | | |
| HMIX2 | 2021 Dec. 20 | NJ | 16.6 | 625,507 | 0.53 | None | 21M | Re- | balanced | N/A |
| | | | | | | | (Omicron) | extraction | | |
| | | | | | | | | and re- | | |
| | | | | | | | | sequencing | | |
| HMIX4 | 2021 Dec. 13 | CA | 19.5 | 826,167 | 0.61 | None | 21K | Re- | Omicron | N/A |
| | | | | | | | (Omicron) | extraction | | |
| | | | | | | | | and re- | | |
| | | | | | | | | sequencing | | |
| HMIX5 | 2021 Dec. 13 | PA | 19.2 | 1,164,229 | 0.54 | None | 21M | Re- | balanced | N/A |
| | | | | | | | (Omicron) | extraction | | |
| | | | | | | | | and re- | | |
| | | | | | | | | sequencing | | |
| HMIX6 | 2021 Dec. 13 | CA | 17.4 | 1,998,992 | 0.51 | None | 21M | Re- | balanced | N/A |
| | | | | | | | (Omicron) | extraction | | |
| | | | | | | | | and re- | | |
| | | | | | | | | sequencing | | |
| HMIX7 | 2021 Dec. 13 | PA | 19.3 | 320,813 | 0.43 | B.1.617.2 | 21J | Re- | Delta | N/A |
| | | | | | | | (Delta) | extraction | | |
| | | | | | | | | and re- | | |
| | | | | | | | | sequencing | | |
| HMIX8 | 2021 Dec. 13 | CA | 24.1 | 31,419 | 0.77 | BA.1 | 21K | Re- | Omicron | N/A |
| | | | | | | | (Omicron) | extraction | | |
| | | | | | | | | and re- | | |
| | | | | | | | | sequencing | | |
| HMIX10 | 2021 Dec. 20 | CA | 24.4 | 20,698 | 0.55 | None | 21K | Re- | Omicron | N/A |
| | | | | | | | (Omicron) | extraction | | |
| | | | | | | | | and re- | | |
| | | | | | | | | sequencing | | |
| HMIX11 | 2021 Dec. 20 | FL | 20.4 | 193,976 | 0.54 | None | 21K | Re- | balanced | N/A |
| | | | | | | | (Omicron) | extraction | | |
| | | | | | | | | and re- | | |
| | | | | | | | | sequencing | | |
| HMIX12 | 2021 Dec. 20 | GA | 21. | 359,378 | 0.83 | AY.25 | 21J | Re- | Delta | N/A |
| | | | | | | | (Delta) | extraction | | |
| | | | | | | | | and re- | | |
| | | | | | | | | sequencing | | |
| HMIX13 | 2021 Dec. 20 | MI | 21.1 | 668,533 | 0.51 | B.1.617.2 | 21J | Re- | Delta | N/A |
| | | | | | | | (Delta) | extraction | | |
| | | | | | | | | and re- | | |
| | | | | | | | | sequencing | | |
| HMIX14 | 2021 Dec. 27 | CA | 17.5 | 2,540,144 | 0.50 | None | 21M | Re- | balanced | N/A |
| | | | | | | | (Omicron) | extraction | | |
| | | | | | | | | and re- | | |
| | | | | | | | | sequencing | | |
| HMIX15 | 2021 Dec. 27 | TX | 22.9 | 41,983 | 0.51 | None | 21K | Re- | balanced | N/A |
| | | | | | | | (Omicron) | extraction | | |
| | | | | | | | | and re- | | |
| | | | | | | | | sequencing | | |
| HMIX16 | 2022 Jan. 3 | FL | 18.2 | 103,983 | 0.51 | None | 21J | Re- | Delta | N/A |
| | | | | | | | (Delta) | extraction | | |
| | | | | | | | | and re- | | |
| | | | | | | | | sequencing | | |
| HMIX17 | 2022 Jan. 3 | FL | 21.8 | 354,336 | 0.81 | BA.1.1 | 21K | Re- | Omicron | N/A |
| | | | | | | | (Omicron) | extraction | | |
| | | | | | | | | and re- | | |
| | | | | | | | | sequencing | | |
| HMIX18 | 2022 Jan. 10 | IN | 17.4 | 3,930,358 | 0.44 | None | 21M | Re- | Delta | N/A |
| | | | | | | | (Omicron) | extraction | | |
| | | | | | | | | and re- | | |
| | | | | | | | | sequencing | | |
| HMIX19 | 2022 Jan. 10 | OK | 19.9 | 1,121,368 | 0.54 | None | 21M | Re- | balanced | N/A |
| | | | | | | | (Omicron) | extraction | | |
| | | | | | | | | and re- | | |
| | | | | | | | | sequencing | | |
| HMIX20 | 2022 Jan. 10 | GA | 19.7 | 692,308 | 0.49 | None | 21M | Re- | balanced | N/A |
| | | | | | | | (Omicron) | extraction | | |
| | | | | | | | | and re- | | |
| | | | | | | | | sequencing | | |
| RECOMB1 | 2022 Jan. 10 | MA | 22.5 | 8,639 | 1.00 | None | 21K | Re- | N/A | 22,204 |
| | | | | | | | (Omicron) | extraction | | to |
| | | | | | | | | and re- | | 22,578 |
| | | | | | | | | sequencing | | |
| RECOMB2 | 2022 Feb. 7 | MA | 20.5 | 240,742 | 1.00 | None | 21K | Re- | N/A | 19,220 |
| | | | | | | | (Omicron) | extraction | | to |
| | | | | | | | | and re- | | 21,618 |
| | | | | | | | | sequencing | | |

Upon filtering for samples where the median alternate allele fraction was less than 0.85, twenty-one samples were identified that were likely to be co-infected with Delta and Omicron variants. Of these, 19 samples were validated the results by RNA re-extraction and re-sequencing. Additional samples were validated using an orthogonal genotyping assay.

Specimens identified as coinfections or recombinants were verified by reprocessing from the original specimen. The process was replicated as described above; however, the hybridization probe panel was substituted. The IDT COVID-19 Capture Panel was replaced with the Respiratory Virus Research Panel (Twist Biosciences), while all other reagents remained the same.

The results replicated in 18 of the 19 samples, shown in FIG. 7B, FIG. 7C, and Table 4. Shown in FIG. 7B are example graphs representing the alternate allele fraction for each mutation. Forty-seven mutations were plotted in order of their position on the SARS-COV-2 genome from 5′ to 3′. Genes are separated by dashed vertical lines. Sixteen mutations specific to Delta are represented. Nineteen mutations specific to Omicron and shared by all its sub-lineages are represented. Five mutations specific to BA.1 (and BA.1.1) are shown. The defining BA. 1.1 mutation is also shown, and 6 mutations specific to BA.2 are shown.

The graphs for all 18 coinfections are illustrated in FIG. 7C. Shown here are graphs representing the Alternate Allele Fraction for each mutation. 35 mutations are plotted in order of their position on the SARS-COV-2 genome from 5′ to 3′. Genes are separated by dashed vertical lines. 18 mutations specific to Delta are represented: ORFIA:A1306S, ORFIA:P2046L, ORFIA:P2287S, ORFIA:V2930L, ORFIA:T3646A, ORF1B:G662S, ORF1B:P1000L, ORF1B:1918V, S:T19R, S:L452R, S:P681R, S:D950N, ORF3A:S26L, M:182T, ORF7A:V82A, ORF7A:T1201, N:D63G, N:R203M. 16 mutations specific to Omicron are represented: ORF1A:P3395H, ORFIB:11566V, S:G339D, S:S375F, S:E484A, S:Q498R, S:H655Y, S:N764K, S:D796Y, S:N969K, E:T9I, M:Q19E, M:A63T, N:P13L, N:DEL31-33, N:R203K. The S:R346K characteristic of Omicron BA. 1.1 is represented as well.

On average, about 1 in 800 positive samples had a coinfection of Delta and Omicron between Dec. 6, 2021, and Jan. 16, 2022. Using the fraction of sequencing reads that mapped to mutations in either Delta or Omicron as a proxy, the fraction of Delta and Omicron virions in a given sample appeared similar (between 40 and 60%) in 8 out of 18 coinfections, seen in FIG. 7B and FIG. 7C.

Here, the Delta variant was higher than the Omicron variant in five coinfection samples, while the Omicron variant was higher than the Delta variant in the remaining five. This can be seen in FIGS. 7B and 7C. Summarized in FIG. 7D, the fraction of Delta and Omicron virions in each sample were similar in the replicates despite re-extraction. FIG. 7D illustrates the difference of Delta fraction and Omicron fraction plotted for the initial sample and the replicate sequenced after a new RNA extraction. Dashed lines connect identical samples.

Shown in FIG. 7E, and FIG. 7F, there was little correlation between the date of coinfection and the dominant variant, or between the viral load in the nose and the dominant variant. FIG. 7E illustrates the difference of Delta fraction and Omicron fraction for the initial sample is plotted against the collection week of the sample. FIG. 7F illustrates the viral load in coinfections. The Cq of each sample, grouped by the dominant variant, is shown. The first column are the samples (n=5) with a majority of Omicron; the second column are the samples (n=8) with a balanced mix of Delta and Omicron; the third column are the samples (n=5) with a majority of the Delta variant. Cq is based on the qRT-PCR assay performed on samples collected from the anterior nares of patients. Whiskers denote the median and 95% Confidence Interval.

### Example 3. Detection of Within-Host Recombination of Delta and Omicron Genomes

A subset of host cells in a coinfection contained both variants, and therefore had the potential to generate recombinants. If these recombinants were replication competent and replicated to high enough titers, they would be detected in sequencing output, manifesting as a change in allele fraction of defining mutations near the recombination breakpoint.

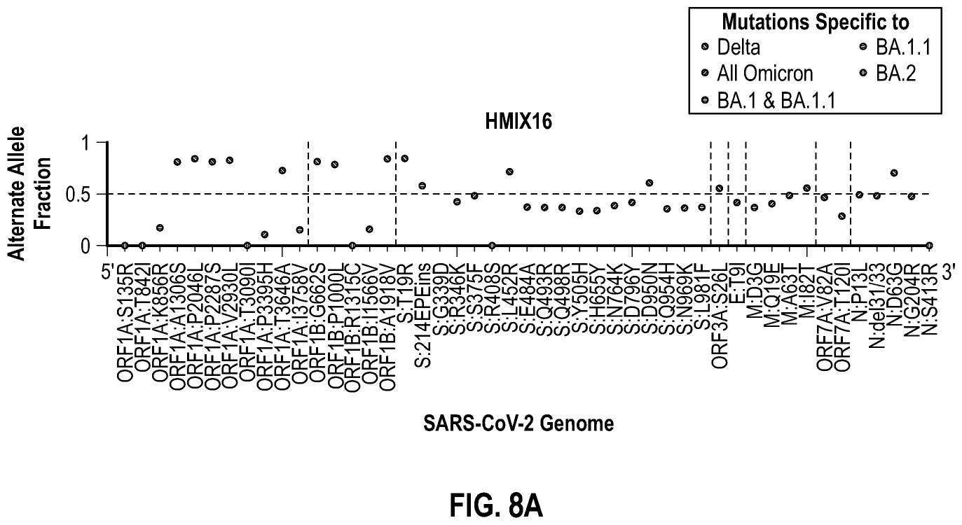

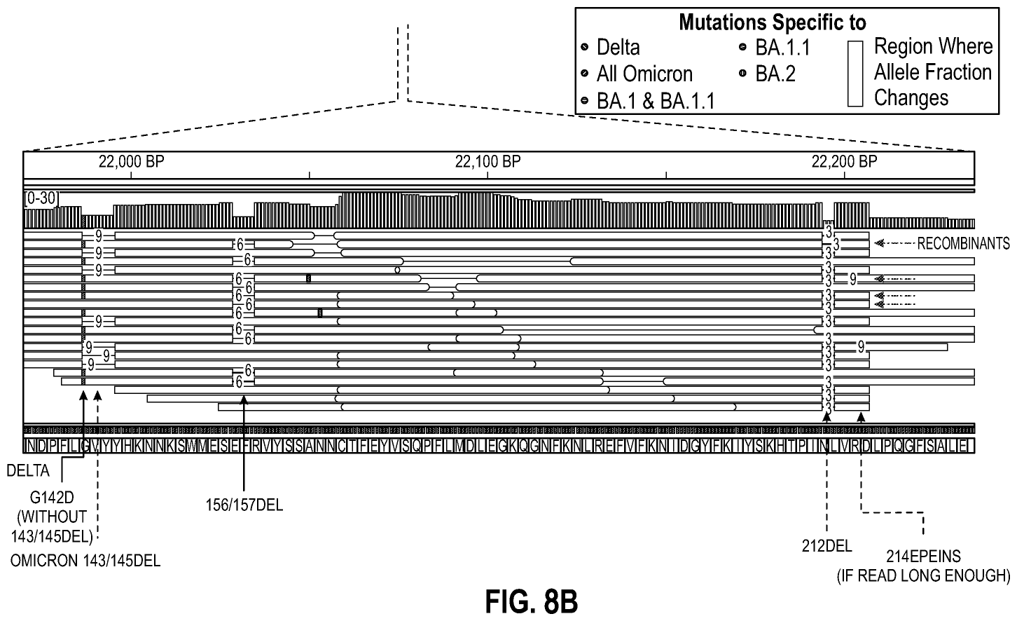

Shown in FIG. 8A, HMIX16 exhibited this characteristic. FIG. 8A depicts evidence of Delta, Omicron, and recombinant read pairs in HMIX16. FIG. 8A illustrates a graph representing the Alternate Allele Fraction for each mutation of sample HMIX16. Forty-seven mutations are plotted in order of their position on the SARS-COV-2 genome from 5′ to 3′. Genes are separated by dashed vertical lines. Sixteen mutations specific to Delta are represented. Nineteen mutations specific to Omicron and shared by all its sub-lineages are represented. Five mutations specific to BA.1 (and BA.1.1) are represented. One BA.1.1 mutation and 6 mutations specific to BA.2 are shown. The box represents the region where the alternate allele fraction changes.

With HMIX16, alternative allele fractions for Delta mutations hovered around 0.80 near the 5′ end of the genome but dropped to around 0.50 near the beginning of the S gene and remain at this level until the 3′ end of the genome. This profile suggests the presence of a Delta-Omicron recombinant with a breakpoint preceding the S:214EPEins.

Upon examination of read-pairs sequenced from HMIX16 that spanned mutations unique for Delta and Omicron upstream of S:214EPEins, 4 read-pairs were found that supported a Delta-Omicron recombinant, 7 read-pairs that supported Delta only, and 10 that supported Omicron only, shown in FIG. 8B.

FIG. 8B depicts an IGV view of the alignments for HMIX16, subsampled to only include read pairs where the first in pair covers the S: 156/157del position, and the second in pair covers the S:212del position. Read pairs representing three mutation profiles are seen: (i) supporting Delta mutations only (7 read pairs), (ii) supporting Omicron mutations only (10 read pairs), and (iii) supporting a Delta/Omicron recombinant (4 read pairs).

Specifically, the read pairs that supported a Delta-Omicron recombinant comprise the S: 156/157del mutation of Delta on the 5′ end, and the S:212del of Omicron on the 3′ end. The existence of these three unique mutation profiles presents compelling evidence that a recombinant virus was generated during coinfection with a breakpoint region of 157 base pairs between nucleotide positions 22,036 and position 22, 193. No read-pairs were found supporting Delta-Omicron recombination in the same interval in the other coinfection samples showing that these recombinations remain rare events.

### Example 4. Investigation of Clonal Delta-Omicron Recombinants

Having established both the presence of coinfections occur and evidence of recombination in vivo, samples were sought out that were composed entirely of recombinant virus. In such samples, it was expected that all mutations called would be supported by ˜100% of the reads because the viral population in the sample would be composed of multiple copies of the same variant, rather than a mixture of two.

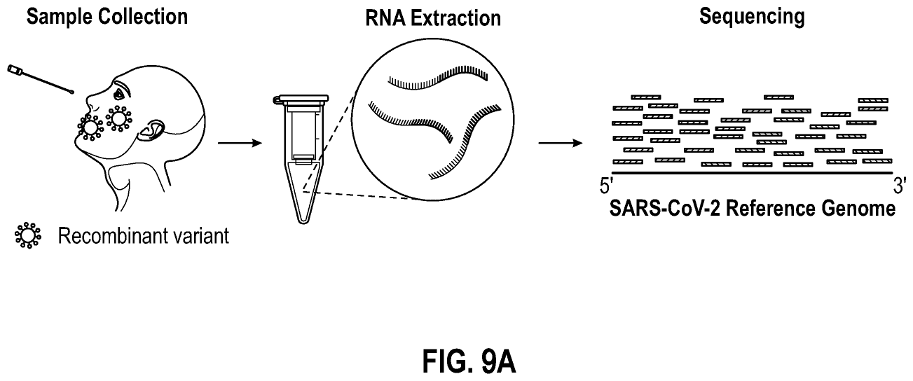

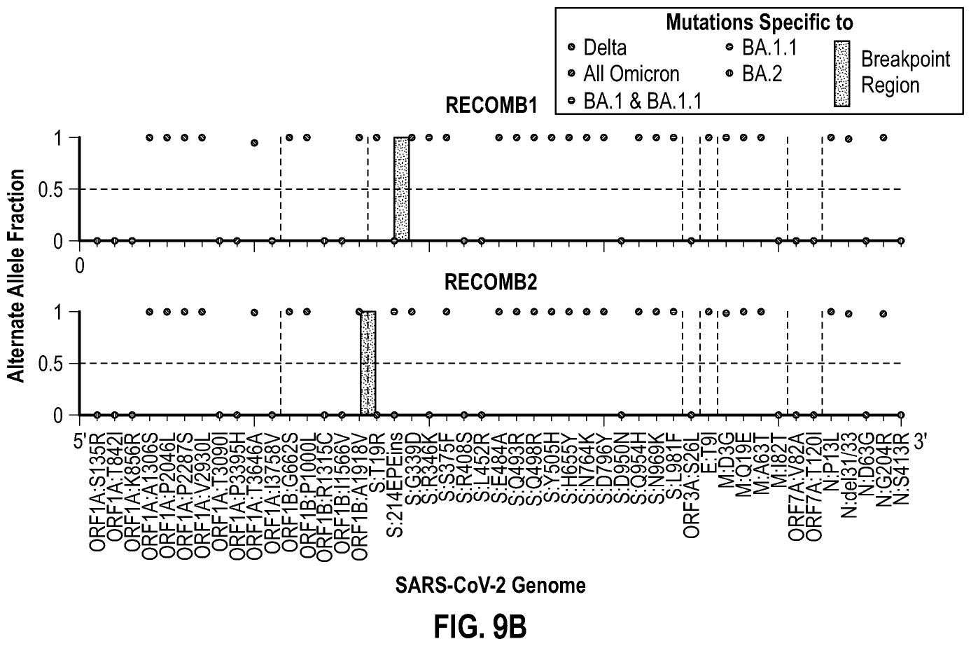

Recombinants with one breakpoint were first looked at, where all mutations identified on the 5′-end of the breakpoint should be characteristic of one variant (e.g., variant A), and all mutations on the 3′-end of the breakpoint should be characteristic of the other variant (e.g., variant B). This is illustrated in FIG. 9A, showing a schematic of the impact of an infection from a virus resulting from the recombination of Delta and Omicron on the sequencing output.

Seven samples were identified that had Delta-specific ORFIA:A1306S at the 5′-end of the genome, and Omicron-specific N:P13L at the 3′-end. One sample had Omicron-specific ORFIA:P3395H at the 5′-end and Delta-specific N:D63G at the 3′-end. Further analysis of these eight genomes showed that only two genomes, RECOMB1 and RECOMB2, had multiple consecutive Delta mutations at the 5′-end while the 3′-end of the genome had all of the Omicron mutations but none of the Delta mutations.

This is illustrated in FIG. 9B, which includes graphs representing the alternate allele fraction for each mutation. Here, forty-seven mutations are plotted in order of their position on the SARS-COV-2 genome from 5′ to 3′. Genes are separated by dashed vertical lines. Sixteen mutations specific to Delta are represented. Nineteen mutations specific to Omicron and shared by all its sub-lineages are represented. Five mutations specific to BA.1 (and BA.1.1) are represented. One BA.1.1 mutation and 6 mutations specific to BA.2 are also shown. The box represents the homologous region where the breakpoint of the recombination was.

Four of the six other genomes had all (5′ to 3′ of the genome) Omicron-specific mutations and the additional Delta ORF1A:A1306S, which was probably acquired independently. The remaining genomes had all of the Delta-specific mutations with one containing an additional Omicron N:P13L, and the other containing Omicron ORF1A:P3395H. These were probably also acquired independently.

The sequences of the two recombinant viruses differed slightly. The breakpoint region of RECOMB1 as 374 bases between nucleotide position 22,204 and position 22,578, while the breakpoint region of RECOMB2 as 2,398 bases between nucleotide position 19,220 and position 21,618, shown in FIG. 9B.

There was a private mutation T19404C in RECOMB2 inside the breakpoint region. RECOMB1 was a recombination between Delta sub lineage AY.119 and Omicron sub lineage BA.1.1. The 5′ Delta end of RECOMB2 was too short for sub lineage classification, but the 3′ end was Omicron sub lineage BA.1. These two samples were both collected in Massachusetts, but the difference in sequence suggests they are unrelated.

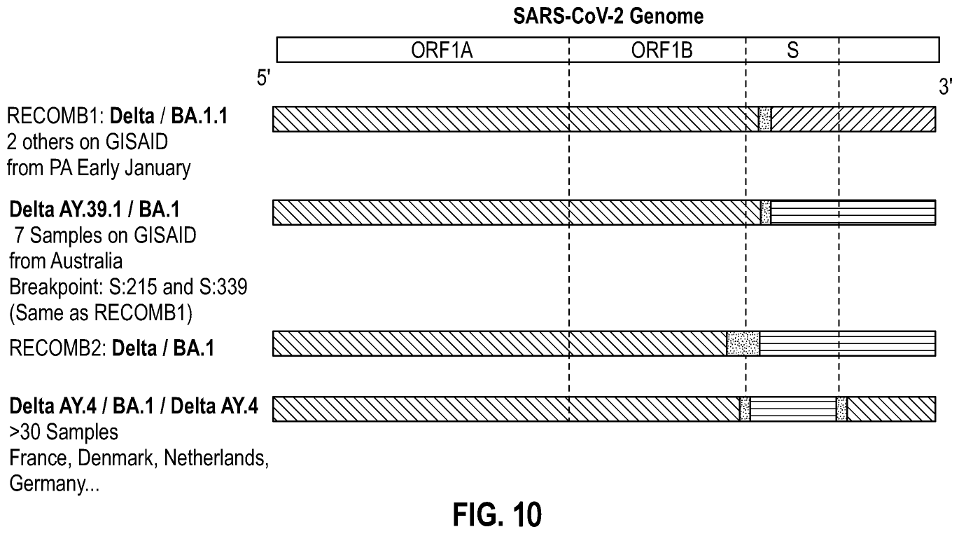

Overall, infections from a recombinant Delta-Omicron virus were rare: 2 out of 10,742 sequences between January 10 and Feb. 13, 2022. Eight other sequences similar to RECOMB1 were reported by the CDC from samples collected in the US from Dec. 31, 2021, to Feb. 12, 2022, shown in FIG. 10.

### Example 5. Validation of Coinfection Cases

Overall, 18 coinfections were identified, one of which displayed evidence of a low Delta-Omicron recombinant viral population. Two independent cases of infection by a Delta-Omicron recombinant virus were identified, where 100% of the viral RNA came from one clonal recombinant. In the three cases, the 5′-end of the viral genome was from the Delta genome, and the 3′-end from Omicron including the majority of the spike protein gene, though the breakpoints were different.

While contamination could lead to the same output as a coinfection, several pieces of evidence discount contamination: re-extraction and re-sequencing these samples led to the same results; the fraction of reads supporting each variant was high in all cases (at least 15%); samples that showed a coinfection were collected and processed on different days, and other samples sequenced on the same plates did not show coinfection; and in one of these coinfections, evidence of recombinant virus at a low but detectable frequency was found, consistent with template-switching during replication in a cell infected with two variants.

Additionally, the data supported chimeric sequences being the cause, rather than technical artifacts as the result were reproducible for samples after re-extracting RNA, and eight other sequences identical or near identical to RECOMB1 were identified in the United States.

### Additional Examples

The following examples are provided, the numbering of which is not to be construed as designating levels of importance:

Example 1 is a method of distinguishing between coinfection or contamination for a biological sample, the biological sample including a first variant of a pathogen and a second variant of the pathogen, wherein the first variant corresponds to a first mutation and the second variant corresponds to a second mutation different than the first mutation, the method comprising: acquiring sequencing data for the biological sample, the sequencing data including a plurality of reads; determining that the biological sample is a mixed sample of the first variant and the second variant based on an alternative allele fraction calculated according to the plurality of reads; searching individual reads of the plurality of reads for recombinant reads that include the first mutation and the second mutation; and determining whether the biological sample is indicative of a coinfection or a contamination, based on an amount of the recombinant reads that indicate the first variant and the second variant.

In Example 2, the subject matter of Example 1 optionally includes wherein determining whether the biological sample is a mixed sample comprises calling an alternative allele at a locus based on either the first mutation or the second mutation.

In Example 3, the subject matter of Example 2 optionally includes wherein determining whether the biological sample is a mixed sample comprises calculating an alternative allele fraction for the called alternative allele.

In Example 4, the subject matter of Example 3 optionally includes wherein calculating an allele fraction for one of the called alternative alleles comprises taking a number of reads of the called alternative allele divided by a total number of reads at the locus.

In Example 5, the subject matter of any one or more of Examples 1˜4 optionally include selecting the first variant and the second variant of the pathogen, wherein selecting the first variant and the second variant comprises retrieving information from a database.

In Example 6, the subject matter of any one or more of Examples 1-5 optionally include wherein determining whether the biological sample is a mixed sample comprises calculating a composite alternative allele fraction based on one or more calculated alternative allele fractions.

In Example 7, the subject matter of Example 6 optionally includes wherein calculating the composite alternative allele fraction determining the median or mean across loci which have an alternative allele fraction of a threshold minimum amount.

In Example 8, the subject matter of Example 7 optionally includes wherein the threshold minimum amount comprises about 0.15.

In Example 9, the subject matter of any one or more of Examples 7-8 optionally include wherein if the composite alternative allele fraction is below a composite alternative allele fraction threshold, the sample is a mixed sample.

In Example 10, the subject matter of any one or more of Examples 7-9 optionally include wherein if the composite alternative allele fraction is above a composite alternative allele fraction threshold, the sample is dominant in one of the first variant and the second variant.

In Example 11, the subject matter of Example 10 optionally includes wherein the composite alternative allele fraction threshold comprises about 0.80.

In Example 12, the subject matter of any one or more of Examples 1-11 optionally include wherein detecting a recombinant pathogen comprises identifying reads or read pairs straddling at least one of the first mutation and the second mutation.

In Example 13, the subject matter of any one or more of Examples 1-12 optionally include ' end to determine whether the first mutation, the second mutation, or combinations thereof are present.

In Example 14, the subject matter of any one or more of Examples 1-13 optionally include wherein detecting a recombinant pathogen comprises identifying reads which start with the first mutation and send with the second mutation.

In Example 15, the subject matter of any one or more of Examples 1-14 optionally include wherein detecting a recombinant pathogen comprises identifying reads that include mutations from both the first variant and the second variant.

In Example 16, the subject matter of any one or more of Examples 1-15 optionally include wherein detecting a recombinant pathogen comprises identifying one or more breakpoints.

Example 17 is a non-transitory machine-readable medium including instructions that, when executed by a processor of a machine, cause the machine to perform operations comprising: acquiring sequencing data for a biological sample, the sequencing data including a plurality of reads; identifying a first variant and a second variant of the biological sample, the first variant corresponding to a first mutation and the second variant corresponding to a second mutation different than the first mutation; determining that the biological sample is a mixed sample based on a composite alternative allele fraction; reviewing individual reads of the plurality of reads for recombinant reads including the first mutation and the second mutation; and determining whether the sample is indicative of a coinfection or a contamination.

In Example 18, the subject matter of Example 17 optionally includes wherein determining whether the biological sample is a mixed sample comprises calling an alternative allele at a locus based on either the first mutation or the second mutation.

In Example 19, the subject matter of Example 18 optionally includes wherein determining whether the biological sample is a mixed sample comprises calculating an alternative allele fraction for the called alternative allele.

In Example 20, the subject matter of Example 19 optionally includes wherein calculating an allele fraction for one of the called alternative alleles comprises taking a number of reads of the called alternative allele divided by a total number of reads at the locus.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method of distinguishing between coinfection and contamination for a biological sample, the biological sample including a first variant of a pathogen and a second variant of the pathogen, wherein the first variant corresponds to a first mutation and the second variant corresponds to a second mutation different than the first mutation, the method comprising:
acquiring sequencing data for the biological sample, the sequencing data including a plurality of reads;
determining that the biological sample is a mixed sample of the first variant and the second variant based on an alternative allele fraction calculated according to the plurality of reads;
searching individual reads of the plurality of reads for recombinant reads that include the first mutation and the second mutation; and
determining whether the biological sample is indicative of a coinfection or a contamination, based on an amount of the recombinant reads that each indicate both the first variant and the second variant.

2. The method of claim 1, wherein determining that the biological sample is a mixed sample comprises calling an alternative allele at a locus based on either the first mutation or the second mutation.

3. The method of claim 2, wherein determining that the biological sample is a mixed sample comprises calculating an alternative allele fraction for the called alternative allele.

4. The method of claim 3, wherein calculating an alternative allele fraction for one of the called alternative alleles is based on a number of reads of the called alternative allele divided by a total number of reads at the locus.

5. The method of claim 1, further comprising selecting the first variant and the second variant of the pathogen, wherein selecting the first variant and the second variant comprises retrieving information from a database.

6. The method of claim 1, wherein determining that the biological sample is a mixed sample comprises calculating at least one of a median alternative allele fraction, a mean alternative allele fraction, a weighted alternative allele fraction, or an average alternative allele fraction, based on one or more calculated alternative allele fractions.

7. The method of claim 6, wherein calculating at least one of a median alternative allele fraction, a mean alternative allele fraction, a weighted alternative allele fraction, or an average alternative allele fraction is based on loci that have alternative allele fractions of at least a threshold minimum fraction.

8. The method of claim 7, wherein the threshold minimum fraction is about 0.15.

9. The method of claim 7, wherein if the calculation based on the alternative allele fraction is below a threshold alternative allele fraction, the sample is a mixed sample.

10. The method of claim 7, wherein if the calculation based on the alternative allele fraction is above a threshold alternative allele fraction, the sample is dominant in one of the first variant and the second variant.

11. The method of claim 10, wherein the threshold alternative allele fraction is about 0.80.

12. The method of claim 1, wherein detecting a recombinant pathogen comprises identifying reads or read pairs straddling at least one of the first mutation and the second mutation.

13. The method of claim 1, wherein determining whether the biological sample is indicative of a coinfection or a contamination comprises walking across the sequence from a 5′ end to a 3′ end to determine whether the first mutation, the second mutation, or combinations thereof are present.

14. The method of claim 1, wherein determining whether the biological sample is indicative of a coinfection or a contamination comprises identifying reads that start with the first mutation and send with the second mutation.

15. The method of claim 1, wherein determining whether the biological sample is indicative of a coinfection or a contamination comprises identifying reads that include mutations from both the first variant and the second variant.

16. The method of claim 1, wherein determining whether the biological sample is indicative of a coinfection or a contamination comprises identifying one or more breakpoints.

17. A non-transitory machine-readable medium including instructions that, when executed by a processor of a machine, cause the machine to perform operations comprising:
acquiring sequencing data for a biological sample, the sequencing data including a plurality of reads;
identifying a first variant and a second variant of the biological sample, the first variant corresponding to a first mutation and the second variant corresponding to a second mutation different than the first mutation;
determining that the biological sample is a mixed sample of the first variant and the second variant based on an alternative allele fraction calculated according to the plurality of reads;
searching individual reads of the plurality of reads for recombinant reads that include the first mutation and the second mutation; and
determining whether the biological sample is indicative of a coinfection or a contamination, based on an amount of the recombinant reads that each indicate both the first variant and the second variant.

18. The non-transitory machine-readable medium of claim 17, wherein determining whether the biological sample is a mixed sample comprises calling an alternative allele at a locus based on either the first mutation or the second mutation.

19. The non-transitory machine-readable medium of claim 18, wherein determining whether the biological sample is a mixed sample comprises calculating an alternative allele fraction for the called alternative allele.

20. The non-transitory machine-readable medium of claim 19, wherein calculating an allele fraction for one of the called alternative alleles comprises taking a number of reads of the called alternative allele divided by a total number of reads at the locus.
